(19) United States
(12) Patent Application Publication
(10) Pub. No.: US 2003/0157190 A1
(43) Pub. Date: Aug. 21, 2003

(54) Method of increasing fertility and athletic performance in horses

(76) Inventor: Gordon L. Woods, Moscow, ID (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/348,344

(22) Filed: Jan. 22, 2003

Related U.S. Application Data
(60) Provisional application No. 60/349,377, filed on Jan. 22, 2002.

(51) Int. Cl. A61K 33/32

(52) U.S. Cl. 424/643

(57) ABSTRACT

Compositions comprising a biologically available and physiologically acceptable zinc salt are administered to horses to increase their fertility and reproduction. These compositions also can be administered to increase horses' athletic performance.

This application claims priority from U.S. provisional application serial No. 60/349,377, filed Jan. 22, 2002.

### FIELD OF THE INVENTION

The present invention relates to methods and compositions for improving reproduction and athletic performance in horses. More specifically, the invention relates to the administration of compositions comprising zinc to horses to increase their fertility and reproduction. These compositions also can be administered to increase horses' athletic performance.

### BACKGROUND OF THE INVENTION

The publications and other materials used herein to illuminate the background of the invention are incorporated by reference herein and for convenience the full citations are provided in the List of References which follows the Examples.

Horses have low reproductive performance in comparison to humans. It is known that sperm motility is correlated to stallion fertility (1). Stallions with lower fertility than the mean overall season fertility in one study had significantly lower mean values for the percentage of motile and progressively motile sperm and for the percentage of morphologically normal sperm. Id.

As a further illustration of difficulties observed in equine reproduction, although human in vitro fertilization has become quite successful, resulting in the birth of over 50,000 babies, equine in vitro fertilization has been successful only rarely; literature reports indicate that, although there have been several thousand attempts, only two foals have been born as a result of in vitro fertilization. All of the reasons for the lack of success for equine in vitro fertilization are not currently known.

There has been very little research to date looking into the relationship between equine fertility and nutrition. Indeed, some researchers have dismissed any consideration of a relationship between fertility and nutrition out of hand; it has been reported that the only dietary requirement for efficient sperm production and satisfactory breeding performance is a balanced diet that maintains the stallion at his optimum weight and that there is no evidence that providing or increasing any nutrient would increase sperm number or quality (2).

Accordingly, there is a need for methods and compositions for increasing reproductive performance in horses.

### SUMMARY OF THE INVENTION

In accordance with the present invention, methods and compositions are provided for increasing horses' reproductive performance. In one embodiment, a method for treating a stallion whose level of sperm motility is below a desired level comprises administering to the stallion a composition comprising a bioavailable and physiologically acceptable zinc salt in a dosage regimen sufficient to increase his sperm motility. In another embodiment of this invention, a method for elevating seminal zinc concentrations in a horse with a seminal zinc deficiency comprises administering to the horse a bioavailable and physiologically acceptable zinc salt in a dosage regimen sufficient to elevate the horse's seminal zinc concentration and minimize or eliminate the zinc deficiency.

A further embodiment of this invention is directed to a method for elevating the concentration of systemic zinc in a horse which comprises administering to a horse a bioavailable and physiologically acceptable zinc salt in a dosage regimen sufficient to minimize, eliminate or prevent a deficiency in said systemic zinc levels.

Another embodiment of this invention comprises a method for enhancing a mare's ability to conceive which comprises administering to the mare prior to an attempt to breed the mare a biologically available and physiologically acceptable zinc salt in an amount sufficient to minimize, eliminate or prevent any zinc deficiency in said mare and thereby increase her ability to conceive.

An additional embodiment is directed to a method for enhancing the birth rate of healthy, live foals, which comprises administering to mares who are pregnant or are to be bred and are or are at risk of becoming zinc deficient a biologically available and physiologically acceptable zinc salt so as to elevate the mares' serum zinc level during their pregnancies so as to eliminate, minimize or prevent said zinc deficiencies and thereby enhance the birth rate of healthy, live foals.

A further embodiment is directed to a method of enhancing the development of a healthy egg by a mare from which an egg will be harvested, such as for a cloning procedure, which comprises administering a biologically available and physiologically acceptable zinc salt to a mare from which an egg will be removed so as to elevate the mare's serum zinc level to eliminate, minimize or prevent any zinc deficiency prior to the formation and removal of her egg.

A related embodiment comprises a method of enhancing the birth rate of a healthy, live foal from an embryo comprising cloned equine DNA which comprises administering to a mare in which an embryo containing cloned DNA has been implanted a biologically available and physiologically acceptable zinc salt in an amount sufficient to minimize, eliminate or prevent a zinc deficiency in said mare during her pregnancy and thereby enhance the birth rate of a healthy, live foal.

It also is believed that equine athletes can suffer from particularly severe zinc deficiencies. A further embodiment of this invention is directed to the administration of a bioavailable and physiologically acceptable zinc salt to horses, including equine athletes, suffering from a zinc deficiency to minimize or eliminate the deficiency and to improve their athletic performance.

### DETAILED DESCRIPTION OF THE INVENTION

It is well-known that zinc is essential for fertility in human males (3, 4, 5). Sperm zinc is high in fertile men (6, 7), and a zinc deficiency causes hypogonadism in men (8), with low serum zinc observed in infertile men (9) and low semen zinc observed in oligospermic men (10). Zinc supplementation has been shown to improve overall sperm parameters (11, 12) and increased sperm motility (13, 14, 15) in zinc deficient men.

Studies also have shown that women are at risk of zinc deficiency during pregnancy (16) and that maternal zinc deficiency is associated with poor fetal growth in both animal and human populations (16,17). Indeed, zinc deficiencies in pregnancy can adversely affect the pregnancy, the baby's birth, and the baby's development. It has been reported that a zinc deficiency during pregnancy is associated with an increased risk for maternal morbidity, especially prolonged gestation, inefficient labor and atonic bleeding at delivery, as well as an increased risk to the fetus, especially malformations and postmaturity (18). Another study found that 60% of the pregnant women participating in the study who gave birth to infants with congenital anomalies had low serum zinc concentrations in the first trimester (19). Women who delivered either before or after normal term had low serum zinc concentrations in the third trimester. Id. A number of studies have reported a significant relation between an indicator of maternal zinc status and birth weight and the incidence of fetal growth retardation (20). One paper reported that women with low serum zinc concentrations were 20-50% more likely to suffer from a variety of maternal and fetal complications of labor and delivery, including fetal distress (21). A review article published in 1993 provides that zinc deficiency has been implicated in infertility, abortions, malformations, fetal intrauterine growth retardation, premature and postmature births, perinatal death, and abnormal deliveries (22). This same paper indicated that zinc therapy in identified low-zinc groups resulted in reduced frequencies of premature birth, placental ablation, perinatal death and postmaturity.

With the exception, in some instances, of pregnant women, healthy humans, especially those whose diets are relatively high in meat and relatively low in fruits and vegetables, tend to have relatively high levels of zinc in their body fluids and tissues (23). In contrast, horses consume diets low in zinc (24).

It now is believed that a significant percentage of horses are zinc deficient and that there is a direct correlation between stallion seminal plasma zinc and sperm motility. It is known that sperm motility is highly correlated with stallion fertility (1).

It also is believed that there is a direct correlation between zinc concentrations in the body fluids of mares and fertility. Evidence supporting this comes from patterns of mare reproductive loss syndrome (MRLS) reported in Kentucky. This syndrome is characterized by a significant number of both early fetal losses and late term aborted fetuses, as well as difficult births and stillbirths, decreased milk production by mares, and foals born weak and unable to thrive (25).

It has been reported that higher than normal incidences of reproductive losses occurred in Kentucky in 1980, 1981 and 2001, in comparison to other years. All three of these years had below normal temperatures in March, as well as frost/freeze dates as late as the third week of April (26). There is evidence that cold, wet springs result in plants with zinc deficiency in comparison to plants grown during warm, dry springs (27). When soils are cold, organic material decomposes slowly or not at all, thereby limiting or preventing zinc release available for uptake by growing crops (28).

From the foregoing, it is hypothesized that during cold, wet springs, zinc levels in pastures of central Kentucky became zinc depleted. Mares grazing exclusively on these pastures did not achieve the levels of zinc in their bodies needed for healthy pregnancies and so suffered high rates of MRLS. This hypothesis is further supported by the fact that mares whose diets were supplemented with hay while out on pasture had a lower or no incidence of MRLS in comparison to mares grazing exclusively on pasture (26).

As used herein, “fertility” or “reproductive performance” is defined to include both the ability to conceive offspring and actual reproductive capacity as measured by the production of healthy offspring. Also as used herein, a “zinc deficiency” is defined as a concentration of zinc which is at least about 15% below desired levels. Many horses have zinc levels which are 20%, or more, below desired levels. Determination of a zinc deficiency can be made easily by measuring the level of zinc in a horse's serum, although the level of zinc in other body fluids can be measured as well. For example, the concentration of zinc in a stallion's seminal plasma can be measured to determine if the stallion has a zinc deficiency.

As noted above, it now is believed that a majority of horses suffer from a zinc deficiency. It is hypothesized that zinc metabolism is similar in horses and in humans and that the relatively low dietary zinc intake in horses causes a primary zinc deficiency in horses, as observed by their significantly lower serum zinc, erythrocyte zinc, sperm zinc and seminal plasma zinc. This low zinc in horses has a negative effect on the blockage of calcium penetration into cells. Calcium concentrations universally control activity of all types of cells in all animal species. Intracellular calcium activates oocyte maturation, sperm capacitation, neurotransmitter release, and skeletal muscle contraction. It now has been determined that calcium levels are significantly lower in equine erythrocytes than in human erythrocytes, and that extracellular calcium is significantly greater in stallions than in men. Although not wishing to be bound by theory, it is hypothesized that the greater blockage of calcium penetration into equine cells decreases intracellular calcium.

Typically, the level of zinc in equine serum is less than or equal to about 660 ng/ml. Although typical, it is believed that this represents a deficiency, as indicated above. Thus, a concentration of about 660 ng/ml or less is considered to be deficient. Desirably, in accordance with this invention, zinc is administered to a horse whose serum zinc concentration is deficient to elevate his or her serum zinc level to within the range of about 760 to about 990 ng/ml, preferably to within the range of about 790 to about 990 ng/ml and more preferably to within the range of about 825 to about 990 ng/ml.

Typically, the level of zinc in the seminal plasma of stallions is less than or equal to about 4000 ng/ml. This level similarly is believed to indicate a zinc deficiency. Desirably, zinc is administered to elevate a stallion's seminal plasma level to within the range of about 4600 to about 6000 ng/ml, preferably to within the range of about 4800 to about 6000 ng/ml and more preferably to within the range of about 5000 to about 6000 ng/ml.

Accordingly, in accordance with one embodiment of the present invention, it now has been found that a bioavailable and physiologically acceptable zinc salt can be administered to horses whose body fluids are zinc deficient to elevate the concentration of zinc in the animal's body fluids and tissues. The zinc is administered so as to minimize or eliminate the deficiency in the level of zinc in body fluids and tissues. Increasing the concentration of zinc in a stallion's serum and seminal plasma leads to an increase in the stallion's sperm motility, and this, in turn, leads to increased fertility and enhanced reproductive performance. Similarly, increasing the concentration of zinc in a mare's serum leads to an increase in the mare's fertility and reproductive performance, which, in turn, leads to an increase in the birth of live, healthy foals. It is believed that low systemic levels of zinc in a mare affects both her production of eggs and their viability as well as the development of the fetus. Thus, in accordance with this invention, a horse's fertility or reproductive performance is enhanced through the administration of a bioavailable and physiologically acceptable zinc salt in an amount sufficient to minimize or eliminate a deficiency in zinc concentration in the horse's body fluids. Furthermore, in addition to minimizing or eliminating a zinc deficiency, a zinc salt also can be administered in an amount sufficient to prevent a deficiency in zinc concentration in a horse's body fluids and tissues.

As noted above, a zinc salt can be administered to a mare which is pregnant or is to be bred to enhance the chances that she will give birth to a live, healthy foal. Such administration can be effected whether the pregnancy is the result of impregnation by a stallion or as the result of a cloning procedure. For example, a zinc salt can be administered to a mare from whom an egg will be harvested to help ensure that the egg removed is a healthy one. Such zinc salt administration typically begins at least about one month prior to the harvesting of an egg. Once an egg is harvested from the ovarian follicles of a “donator” mare, it can be used in accordance with conventional cloning procedures known to persons of skill in the art. For example, the mare's DNA is removed from the egg and replaced with the DNA of the horse to be cloned, then the resultant embryo is implanted in the uterus or fallopian tubes of a “recipient” mare, who may be the same horse who donated the egg or may be a different mare. A zinc salt further is administered to the “recipient” mare during her pregnancy in accordance with the teachings above to enhance the chances of her delivering a healthy, live foal. The zinc salt administration can be effected regardless of the specific cloning procedure employed.

The zinc is administered in the form of a bioavailable and physiologically acceptable zinc salt, including the sulfate, chloride, oxide, gluconate, acetate, citrate, picolinate or stearate salt. Preferred salts include the sulfate, chloride and oxide salts. A single salt or a combination of salts can be used.

The zinc salt can be administered orally or parenterally at a dosage level of about 60 to about 6,000 mg/day. Oral administration can be by capsules, tablets, pastes, suspensions or solutions. If administered in the form of a tablet, the zinc salt can be mixed with one or more lubricants, such as stearic acid or magnesium stearate, flavor ameliorating agents, disintegrating elements, including potato starch and alginic acid, binders, such as gelatin and corn starch, and/or tablet bases, such as lactose, corn starch and sucrose, and then pressed into tablets. Alternatively, the zinc salt can be given in the form of a capsule, prepared by mixing the salt with a pharmaceutically acceptable excipient and then filling gelatin capsules with the mixture in accordance with conventional procedures. The zinc salt also can be administered in the form of a suspension or solution, using a bioavailable and physiologically acceptable liquid carrier, such as water or an oil, or as a paste, using a carrier such as a combination of corn starch and glycerol. If administered orally, the zinc salt typically is administered at a dose of about 600 to about 6000 mg per day.

As an alternative to oral administration, the zinc salt can be administered parenterally, provided in injectable doses of a solution or suspension in a physiologically acceptable diluent with a pharmaceutical carrier. The carrier can comprise water or an oil and also optionally can comprise a surfactant or pharmaceutically acceptable adjuvant. If administered parenterally, the zinc salt typically is administered at a dose of about 60 to about 600 mg/day.

As noted above, zinc deficiencies in horses can be the result of insufficient zinc in their diets. Zinc deficiencies also can be caused or exacerbated by increased zinc excretion from body fluids and tissues during strenuous exercise. Thus, equine athletes, such as racehorses and jumpers, can suffer from particularly severe zinc deficiencies, which can adversely affect the animals' growth or muscular performance. In human athletes, a zinc deficiency has been associated with an inability to maintain plasma glucose and with a tendency to exhibit hypoglycemia. Hypozincemia also has been associated with a higher increase in blood lactate and a lower power output during the exercise period. In a further embodiment of this invention, a bioavailable and physiologically acceptable zinc salt is administered to horses, including equine athletes, to regulate their systemic levels of zinc and to improve their athletic performance. A horse's athletic performance is conventionally determined on the basis of his speed, using conventional, quantifiable, well-defined standards known to persons of skill in the art. Desirably, the zinc salt is administered to increase the level of zinc in the blood or seminal plasma to the levels set forth above. Also desirably, the zinc salt is administered to maintain the zinc concentration within this elevated range even during or immediately after strenuous exercise.

The invention is further illustrated by the following examples, which are provided for illustrative purposes and are not to be construed as limiting.

### EXAMPLE

Experiment 1: Correlation Between Stallion Sperm Motility and Seminal Plasma Zinc

Seven stallions were kept in the absence of light for week one, then were exposed to sunlight for week two. Semen was collected three times during each week. The collected semen was evaluated at intervals of 0, 15, and 30 minutes post-ejaculation using the Hamilton Thorne Research Integrated Visual Optical System (HTM-IVOS) program with a Computer Assisted Semen Analyzer. A pre-warmed counting chamber was charged with 5 μl of raw semen and inserted into the computer.

Experiment 2: Comparison of Serum Zinc and Urine Zinc Levels in Stallions and Humans

Blood and urine was collected from seven healthy stallions. Collections began at 6:00 AM and ended at 8:00 PM. Every hour one syringe of blood was drawn from an indwelling catheter. During the hours of 8:00 AM, 1:00 PM and 7:00 PM blood was drawn every 15 minutes. Alfalfa was provided at 8:00 AM and again at 12:00. Water was free choice during the day. All urine excretion during the day was collected and assigned a time to the closed hour of urination.

Seven healthy non-smoking men ages 30-52 years donated blood and urine. Men fasted from 6:00 PM the evening prior to sampling until breakfast at 8:00 AM of the collection day. Men ate a breakfast of a poppy seed muffin and a 1/2 liter of orange juice. Men ate a chicken salad between the hours of 12:00 PM and 2:00 PM. Two liters of bottled water were provided for each man. Twenty-four blood samples, and eight urine samples (collected at two-hour intervals) were collected for each man.

Experiment 3: Correlation of Seminal Plasma Zinc and Sperm Cell Zinc

Fifty-five mixed breed light horse stallions and fifty-five, healthy, non-smoking men, donated two blood samples for erythrocytes and blood serum.

Erythrocytes and blood serum were handled similarly for stallions and men. Blood samples were collected in two 9 ml heavy metal free syringes. One syringe contained 250 μl of EDTA to obtain erythrocytes. Immediately after collection, the EDTA tube was mixed gently and then centrifuged at 800 G for 15 minutes. Plasma then was removed along with a layer of white blood cells. Erythrocytes then were washed. Three ml of Tris (Tris hydroxymethyl aminomethane hydrochloride) buffered saline (pH 7.0) were added to the erythrocytes and mixed gently and the centrifuged at 800 G for 15 minutes. Supernatant was removed and the process was repeated once more. After washing, the erythrocytes were re-suspended in a volume of saline equal to the original volume of blood.

A second syringe was used to collect blood serum. After collection, whole blood samples were stored at room temperature (21° C.) until blood clots contracted. Tubes were then centrifuged at 800 G for 15 minutes. Serum was subsequently removed with a disposable polypropylene transfer pipette and placed into 1.7 ml micro centrifuge tubes. Processed blood samples were stored in a −80° C. freezer.

Urine was collected during a 1-day period for stallions and men in Experiment II. Each time a stallion urinated, the collection device was removed and a new one was secured. Immediately after collection throughout the day each urine sample was transferred into 60 ml polypropylene bottles that previously had been washed with Radiacwash. Samples were stored in a −80° C. freezer.

Urine samples from men were collected in heavy metal free polypropylene cups, previously rinsed with Radiacwash. Urine was refrigerated as it was collected. At the end of the day, samples were retrieved and processed. Urine from each cup was transferred into 30 ml polypropylene bottles and stored in a −80° C. freezer.

Results

Progressive motility (r=0.65; p=0.0019), total motility (r=0.71; p=0.00042), average path velocity (r=0.72; p=0.019), progressive velocity (r=0.71; p=0.022) and curvilinear velocity (r=0.72; p=0.019) were correlated with seminal plasma zinc in the 7 stallions. It is known that these five measures are correlated with fertility; this is the first time that zinc in seminal plasma has been correlated with these measures.

Serum zinc was 1.8 times lower (456 ng/ml vs. 842 ng/ml; p<0.001) and urine zinc was 3.5 times lower (175 ng/ml vs. 606 ng/ml; p=0.002) for 7 stallions vs. 7 men, respectively (experiment 2). Serum zinc was 1.5 times lower (654 ng/ml vs. 997 ng/ml; p<0.001), erythrocyte zinc was 2.2 times lower (2053 ng/ml vs. 4608 ng/ml; p<0.001), sperm cell zinc was 10.2 times lower (4203 ng/ml vs. 42744 ng/ml; p<0.001) and seminal plasma zinc was 51.9 times lower (1333 ng/ml vs. 69298 ng/ml; p<0.001) for the 55 stallions vs. the 55 men, respectively (experiment 3). The significantly lower serum, urine, erythrocyte, sperm cell, and seminal plasma zinc for stallions vs. men documents that the concentration of zinc is consistently lower in stallions than in men.

Seminal plasma zinc was correlated with sperm cell zinc (r=0.57; p<0.001) (experiment 3). Therefore, the lower seminal plasma zinc, the lower sperm cell zinc. Sperm cell zinc was negatively correlated with age. Age-related changes occur in semen parameters of stallions; therefore, declining sperm cell zinc with increasing stallion age negatively influences the fertility of aging stallions.

TABLE 1
Zinc (ng/ml) in the Serum and Urine
of 7 Men and 7 Stallions (Experiment II)

| zinc | Stallions | Men | (p-value) |
|---|---|---|---|
| Zinc | | | |
| Serum | 456 ± 55 | 842 ± 93 | (<0.001) |
| Urine | 175 ± 92 | 606 ± 299 | (0.002) |

TABLE 2
Zinc (ng/ml) in the
Erythrocytes, Serum, Sperm Cells, and Seminal Plasma
of 55 Men and 55 Stallions (Experiment III)

| zinc | Stallions | Men | (p-value) |
|---|---|---|---|
| Zinc | | | |
| Erythrocytes | 2,053 ± 537 | 4,608 ± 1,353 | (<0.001) |
| Serum | 654 ± 204 | 997 ± 237 | (<0.001) |
| Sperm Cells | 4,203 ± 2,231 | 42,744 ± 24,860 | (<0.001) |
| Seminal | 1,333 ± 710 | 69,298 ± 76,567 | (<0.001) |
| Plasma | | | |

TABLE 3
Serum Zinc (ng/ml) in 55 Men (bold)
and 55 Stallions (Experiment III)
Serum Zinc

| | | | |
|---|---|---|---|
| 2080 | 1433 | 1424 | |
| 1390 | 1287 | 1282 | |
| 1209 | 1209 | 1203 | |
| 1180 | 1175 | 1158 | |
| 1149 | 1137 | 1124 | |
| 1121 | 1116 | 1106 | |
| 1101 | 1054 | 1045 | |
| 1044 | 1034 | 1031 | |
| 1025 | 1009 | 998 | |
| 987 | 977 | 976 | |
| 969 | 962 | 957 | |
| 952 | 928 | 926 | |
| 914 | 908 | 906 | |
| 885 | 884 | 880 | |
| 878 | 876 | 876 | n = 93 |
| 864 | 859 | 856 | |
| 856 | 853 | 843 | |
| 839 | 837 | 830 | |
| 830 | 827 | 814 | |
| 808 | 801 | 795 | |
| 795 | 790 | 769 | |
| 765 | 730 | 730 | |
| 729 | 728 | 723 | |
| 698 | 694 | 687 | |
| 686 | 683 | 683 | |
| 678 | 668 | 667 | |
| 653 | 651 | 641 | |
| 634 | 628 | 606 | |
| 602 | 591 | 585 | |
| 578 | 574 | 568 | |
| 565 | 556 | 553 | |
| 537 | 536 | 535 | |
| 531 | 519 | 514 | |
| 472 | 456 | 454 | n = 17 |
| 447 | 427 | 383 | |
| 377 | 372 | 371 | |
| 367 | 337 | | |

X = 825
S.D. = 280

### REFERENCES

1. Jasko, D. J. et al., “Comparison of spermatozoal movement and semen characteristics with fertility in stallions: 64 cases (1987-1988),” J Am Vet Med Assoc, 200(7):979-985 (1982). PM:1577655.

2. Umphenour, N. W. and Steiner, J. V., “Breeding Management of the Thoroughbred Stallion,” Equine Breeding Management and Artificial Insemination. Samper, J. C. (W.B. Saunders Company, Philadelphia, 2000), chap. 5.

3. Favier, A. E., “The role of zinc in reproduction. Hormonal mechanisms,” Biol Trace Elem Res, 32:363-382 (1992). PM:1375078.

4. Hidiroglou, M. and Knipfel, J. E., “Zinc in mammalian sperm: a review,” J. Dairy Sci, 67(6):1147-1156 (1984). PM:6378991.

5. Bedwal, R. S. and Bahuguna, A., “Zinc, copper and selenium in reproduction,” Experientia, 50(7): 626-640 (1994). PM:8033970.

6. Kvist, U. et al., “Zinc in sperm chromatin and chromatin stability in fertile men and men in barren unions,” Scand J. Urol Nephrol, 22(1):1-6 (1988). PM:3387905.

7. Kvist, U. et al., “Sperm nuclear zinc, chromatin stability, and male fertility,” Scanning Microsc, 1(3):1241-1247 (1987). PM:3659861.

8. Prasad, A. S. et al., “Zinc status and serum testosterone levels of healthy adults,” Nutrition, 12(5):344-348 (1996). PM:8875519.

9. Madding, C. I. et al., “Serum and semen zinc levels in normozoospermic and oligozoospermic men,” Ann Nutr Metab, 30(4):213-218 (1986). PM:3752921.

10. Robak-Cholubek, D. et al., “Zinc levels in seminal plasma and sperm density,” Ginekol Pol, 69(6):490-493 (1998). PM:9695369.

11. Takihara, H. et al., “Effect of low-dose androgen and zinc sulfate on sperm motility and seminal zinc levels in infertile men,” Urology, 22(2):160-164 (1983). PM:6879889.

12. Antoniou, L. D. et al., “Reversal of uraemic impotence by zinc,” Lancet, 2(8044):895-898 (1977). PM:72240.

13. Caldamone, A. A. et al., “Seminal zinc and male infertility,” Urology, 13(3):280-281 (1979). PM:442346.

14. Magnus, 0. et al., “Effects of manganese and other divalent cations on progressive motility of human sperm,” Arch Androl, 24(2):159-166 (1990). PM:2327825.

15. Omu, A. e. et al., “Treatment of asthenozoospermia with zinc sulphate: andrological, immunological and obstetric outcome,” Eur J. Obstet Gynecol Reprod Biol, 79(2):179-184 (1998). PM:9720838.

16. Swanson, C.A., King J. C., “Zinc and pregnancy outcome,” Am J Clin Nutri 46:763-71 (1987).

17. Apgar, J., “Zinc and reproduction,” Annu Rev Nutr 5:43-68 (1985).

18. Jameson, S., Clinical Applications of Recent Advances in Zinc Metabolism, Alan R. Liss, New York, p. 39 (1982).

19. Prasad, A. S. and Cossak, Z. T., “Zinc supplementation and growth in sickle cell disease,” Ann. Intern. Med., 100, 367-371 (1984).

20. King, J.C., “Determinants of maternal zinc status during pregnancy,” Am J. Clin Nutri, 71:1334S-43S (2000) (suppl).

21. Mukherjee, J. M. D., Sandstead, H. H., Ratnaparkhi, M. V., Johnson, L. K., Milne, D. D., Selling, H. P., “Maternal zinc, iron, folic acid, and protein nutriture and outcome of human pregnancy,” Am J. Clin Nutr, 40:496-507 (1984).

22. Jameson, S., “Zinc status in pregnancy: the effect of zinc therapy on perinatal mortality, prematurity, and placental ablation,” Ann NY Acad Sci, 678: 178-92 (1993).

23. Barceloux, D. G., “Zinc,” J. Toxicol Clin Toxicol, 37(2):279-292 (1999). PM:10382562.

24. Hintz, H. F., “Feeds and Feeding,” The Horse, Evans, W. J. et al. (W. H. Freeman and Company, New York, 1990), chap. 7.

25. University of Kentucky, College of Agriculture, Veterinary Science, Mare Reproductive Loss Syndrome Index Page, news release. http://www.uky.edu/Agriculture/VetScience/mrls/may07.htm.

26. University of Kentucky, College of Agriculture, Veterinary Science, Mare Reproductive Loss Syndrome Index Page, Research Status Report & Executive Summary. http://www/uky.du/Aqriculture/VetScience/mrsl/summary914.htm.

27. Follett, R. H., Westfall, D. G., Zinc and Iron Deficiencies, Colorado State University Cooperative Extension, Number 0.545 (1999)

28. Rehm, G., and Michael, S., Zinc for crop production, Communication and Educational/Technology Services, University of Minnesota Extension Service, MI-0720-GO (1997).

1. A method of increasing the fertility of a horse which comprises administering to a horse in need of such treatment a bioavailable and physiologically acceptable zinc salt in an amount sufficient to increase the fertility of the horse.

2. The method of claim 1, wherein the horse is a stallion and the administration of said zinc salt results in increased sperm motility of said stallion.

3. The method of claim 1, wherein the horse is a mare and the administration of said zinc salt increases the mare's ability to give birth to a live, healthy foal.

4. A method for treating a stallion whose sperm motility is below a desired level which comprises administering to the stallion a bioavailable and physiologically acceptable zinc salt in an amount sufficient to increase his sperm motility to said desired level.

5. A method for increasing the rate of conception in mares which comprises administering to said mare prior to a breeding attempt a biologically available and physiologically acceptable zinc salt in an amount sufficient to minimize, eliminate or prevent a zinc deficiency in said mare and thereby increase her ability to conceive.

6. A method for enhancing the production of healthy eggs in a mare's ovaries which comprises administering to a mare of reproductive age a biologically available and physiologically acceptable zinc salt in an amount sufficient to minimize, eliminate or prevent a zinc deficiency in said mare and thereby enhance her ability to produce healthy eggs.

7. A method for increasing the frequency with which an equine pregnancy results in the birth of a healthy, live foal which comprises administering to a pregnant mare a bioavailable and physiologically acceptable zinc salt so as to elevate the mare's serum zinc level during her pregnancy to a level which enhances her ability to give birth to a healthy, live foal.

8. The method of claim 7, wherein said pregnancy results from implanting in said mare an embryo containing DNA of a horse to be cloned.

9. A method for elevating the concentration of zinc in the body fluids and tissues of a horse which comprises administering to a horse suffering from a zinc deficiency in his or her body fluids and tissues a bioavailable and physiologically acceptable zinc salt in a dosage regimen sufficient to eliminate or minimize said zinc deficiency.

10. The method of claim 9, wherein said zinc deficiency comprises a serum zinc concentration at least 15% below a desired level.

11. The method of claim 9, wherein said serum zinc concentration is elevated to within the range of about 760 to about 990 ng/ml.

12. The method of claim 11, wherein said serum zinc concentration is elevated to within the range of about 790 to about 990 ng/ml.

13. The method of claim 12, wherein said serum zinc concentration is elevated to within the range of about 825 to about 990 ng/ml.

14. The method of claim 9, wherein said zinc deficiency comprises a seminal plasma zinc concentration at least 15% below a desired level.

15. The method of claim 14, wherein said seminal plasma zinc concentration is elevated to within the range of about 4600 to about 6000 ng/ml.

16. The method of claim 15 wherein said seminal plasma zinc concentration is elevated to within the range of about 4800 to about 6000 ng/ml.

17. The method of claim 16, wherein said seminal plasma zinc concentration is elevated to within the range of about 5000 to about 6000 ng/ml.

18. A method for elevating the concentration of zinc in the body fluids and tissues of a horse which comprises administering to a horse a bioavailable and physiologically acceptable zinc salt in a dosage regimen sufficient to prevent a zinc deficiency in his or her body fluids and tissues.

19. A method of increasing the athletic performance of a horse, which comprises administering a bioavailable and physiologically acceptable zinc salt to said horse in an amount sufficient to increase said horse's exercise performance.

20. The method of claim 19, wherein said horse suffered from a zinc deficiency, and the zinc salt is administered so as to eliminate or minimize said deficiency.

21. The method of claim 1, 4, 5, 6, 7, 9, 18 or 19, wherein said zinc salt comprises the sulfate, chloride, oxide, gluconate, acetate, citrate, picolinate or stearate zinc salt.

22. The method of claim 1, 4, 5, 6, 7, 9, 18 or 19, wherein said zinc salt is administered at a daily dose of about 60 to about 6000 mg/day.

23. The method of claim 1, 4, 5, 6, 7, 9, 18 or 19, wherein said zinc salt is administered orally or parenterally.

24. The method of claim 23, wherein said zinc salt is administered orally in a daily dose within the range of about 600 to about 6000 mg/day.

25. The method of claim 23, wherein said zinc salt is administered parenterally in a daily dose within the range of about 60 to about 600 mg/day.
